(19) United States
(12) Patent Application Publication
(10) Pub. No.: US 2026/0183479 A1
(43) Pub. Date: Jul. 2, 2026

(54) MEDICAMENT DELIVERY SYSTEM, METHODS OF ADJUSTING AND PERSONALIZING MEDICAMENT DELIVERY, AND RELATED SYSTEMS AND METHODS

(71) Applicant: Insulet Corporation, Acton, MA (US)

(72) Inventors: Joon Bok Lee, Acton, MA (US); William J. Whiteley, Knoxville, TN (US); Ashutosh Zade, San Diego, CA (US); Jason O'Connor, Acton, MA (US)

(21) Appl. No.: 19/438,144

(22) Filed: Dec. 31, 2025

Related U.S. Application Data
(60) Provisional application No. 63/740,755, filed on Dec. 31, 2024.

(51) Int. Cl. A61M 5/142

(52) U.S. Cl. CPC A61M 5/142; A61M 2005/14208; A61M 2202/0007; A61M 2202/04; A61M 2205/3303; A61M 2205/3379; A61M 2205/502; A61M 2230/005; A61M 2230/201

(57) ABSTRACT

A system for administration of medicament to a user-body, the system comprising an automated medicament delivery device configured to deliver medicament to a user utilizing an AID algorithm using a set of initial input parameter values, receive a user input selecting at least one desired therapy parameter value, receive an additional user input enabling optional adjustments to input parameter values of an AID algorithm at least partially controlling delivery of the medicament, adjust one or more input parameters values of the set of initial input parameter values of the AID algorithm to create a set of adjusted input parameter values, generate a request to adjust administration of medicament to a user via the automated medicament delivery device utilizing at least a portion of the AID algorithm using the set of adjusted input parameter values, and deliver medicament to the user according the generated request.

### CROSS-REFERENCE TO RELATED APPLICATIONS

This application claims the benefit under 35 U.S.C. § 119(e) of U.S. Provisional Patent Application Ser. No. 63/740,755, filed Dec. 31, 2024, the disclosure of which is hereby incorporated herein in its entirety by this reference.

### TECHNICAL FIELD

The present disclosure generally relates to medicament delivery systems. More particularly, the present disclosure relates to automated medicament delivery algorithms and personalizing the algorithms.

### BACKGROUND

Automated medicament delivery devices (“AMD,” e.g., an Automated Insulin Delivery (AID) device, without limitation) are often used to administer medicaments to the body of a patient via a cannula inserted into the body to treat medical conditions (e.g., Type 1 Diabetes, without limitation).

Target glucose concentration (i.e., target blood glucose level) is a relatively intuitive (e.g., easily understood) setting in AID systems that allows users (e.g., patients) to control medicament administration. Specifically, a target glucose concentration setting can be understood as an increase in medicament (e.g., insulin) delivery above basal levels if predicted glucose concentrations exceed a target glucose concentration, and a reduction or suspension of medicament (e.g., insulin) delivery if predicted glucose concentrations fall below the target glucose concentration.

### BRIEF SUMMARY

One or more embodiments include a system for administration of medicament to a user-body, the system includes an analyte sensor and an automated medicament delivery device. The automated medicament delivery device includes at least one processor and at least one non-transitory computer-readable storage medium storing instructions thereon that, when executed by the at least one processor, cause the automated medicament delivery device to receive a user input selecting at least one desired therapy parameter value, receive an additional user input enabling optional adjustments to input parameter values of an AID algorithm at least partially controlling delivery of medicament via the automated medicament delivery device, responsive to receiving the additional user input, adjust one or more input parameters values of a set of initial input parameter values of the AID algorithm to create a set of adjusted input parameter values, and generate a request to adjust administration of medicament to a user via the automated medicament delivery device utilizing at least a portion of the AID algorithm using the set of adjusted input parameter values.

The at least one desired therapy parameter value may include a target blood glucose level.

The at least one desired therapy parameter value may include at least one of a target pre-prandial glucose level, a TDI (Total Daily Insulin) value, a basal rate for a specific period of time, or a daily basal rate.

The set of initial input parameter values of the AID algorithm may cause the AID algorithm to exhibit a first level of aggressiveness and the set of adjusted input parameter values may cause the AID algorithm to exhibit a second, different level of aggressiveness.

The second, different level of aggressiveness may include an increased level of aggressiveness relative to the first level of aggressiveness.

Causing the automated medicament delivery device to deliver medicament to the user according to the generated request may include causing the automated medicament delivery device to deliver medicament to the user via least one of basal dosing or a bolus dose determined via the AID algorithm using the set of adjusted input parameter values.

Adjusting one or more input parameters values of the set of initial input parameters values of the AID algorithm may include adjusting each of a Q:R Ratio, a TDI (Total Daily Insulin), and an insulin-on-board (IOB) constraint duration of insulin action value.

The system for administration may also include instructions that, when executed by the at least one processor, cause the automated medicament delivery device to, prior to receiving the additional user input enabling optional adjustments to input parameter values of the AID algorithm, deliver medicament to the user utilizing the AID algorithm using the set of initial input parameter values.

The system for administration may also include instructions that, when executed by the at least one processor, cause the automated medicament delivery device to deliver medicament to the user according to the generated request.

Responsive to receiving the additional user input, adjust one or more input parameters values of a set of initial input parameters of the AID algorithm to create a set of adjusted input parameter values may include automatically adjusting one or more input parameters values of the set of initial input parameters values of the AID algorithm based at least partially on the selected at least one desired therapy parameter value and without additional user input.

Adjusting one or more input parameters values of the set of initial input parameters values of the AID algorithm may include adjusting at least one of a Q:R Ratio, a TDI (Total Daily Insulin) value, an insulin-on-board (IOB) constraint duration of insulin action value, an insulin sensitivity factor (ISF) value, a carbohydrate ratio (CR), an amount of daily dose of long-acting insulin (LAI), amounts of anticipated bolus doses, carb count of meals, or a basal rate.

The initial input parameter values may include one or more of a Q:R Ratio, a TDI (Total Daily Insulin) value, an insulin-on-board (IOB) constraint duration of insulin action value, an insulin sensitivity factor (ISF) value, a carbohydrate ratio (CR), an amount of daily dose of long-acting insulin (LAI), amounts of anticipated bolus doses, carb count of meals, a basal rate, or a target blood glucose level.

Some embodiments may include a method for managing medicament delivery to a user. The method may include receiving, via a handheld electronic computing device, a user input selecting at least one desired therapy parameter value, receive, via the handheld electronic computing device, an additional user input enabling optional adjustments to input parameter values of an AID algorithm at least partially controlling delivery of medicament via an automated medicament delivery device, responsive to receiving the additional user input, adjust one or more input parameters values of a set of initial input parameter values of the AID algorithm to create a set of adjusted input parameter values, and generate a request to adjust administration of medicament to a user via the automated medicament delivery device utilizing at least a portion of the AID algorithm using the set of adjusted input parameter values.

The at least one desired therapy parameter value may include a target blood glucose level.

The at least one desired therapy parameter value may include at least one of a target pre-prandial glucose level, a TDI (Total Daily Insulin) value, a basal rate for a specific period of time, or a daily basal rate.

The set of initial input parameter values of the AID algorithm may cause the AID algorithm to exhibit a first level of aggressiveness, and the set of adjusted input parameter values may cause the AID algorithm to exhibit a second, different level of aggressiveness.

The second, different level of aggressiveness may include an increased level of aggressiveness relative to the first level of aggressiveness.

The method may further include causing the automated medicament delivery device to, prior to receiving the additional user input enabling optional adjustments to input parameter values of the AID algorithm, deliver medicament to the user utilizing the AID algorithm using the set of initial input parameter values.

Adjusting one or more input parameters values of the set of initial input parameters values of the AID algorithm may include adjusting one or more of a glucose cost coefficient, an insulin cost coefficient, a Q:R Ratio, a TDI (Total Daily Insulin), and an insulin-on-board (IOB) constraint duration of insulin action value.

One or more embodiments include a system for administration of medicament to a user-body, the system includes an analyte sensor and an automated medicament delivery device. The automated medicament delivery device includes at least one processor and at least one non-transitory computer-readable storage medium storing instructions thereon that, when executed by the at least one processor, cause the automated medicament delivery device to deliver medicament to a user utilizing an AID algorithm using a set of initial input parameter values, receive a user input selecting at least one desired therapy parameter value, receive an additional user input enabling optional adjustments to input parameter values of an AID algorithm at least partially controlling delivery of medicament via the automated medicament delivery device, responsive to receiving the additional user input, adjust one or more input parameters values of the set of initial input parameter values of the AID algorithm to create a set of adjusted input parameter values, generate a request to adjust administration of medicament to a user via the automated medicament delivery device utilizing at least a portion of the AID algorithm using the set of adjusted input parameter values, deliver medicament to the user according the generated request.

Other technical features may be readily apparent to one skilled in the art from the following figures, descriptions, and claims.

Within the scope of this application, it should be understood that the various aspects, embodiments, examples and alternatives set out herein, and individual features thereof may be taken independently or in any possible and compatible combination. Where features are described with reference to a single aspect or embodiment, it should be understood that such features are applicable to all aspects and embodiments unless otherwise stated or where such features are incompatible.

### BRIEF DESCRIPTION OF THE DRAWINGS

The present disclosure is illustrated and described herein with reference to the various drawings, in which like reference numbers are used to denote like system components/method steps, as appropriate, and in which:

FIG. 1 is a schematic diagram illustrating a medicament delivery system in accordance with one or more examples;

FIG. 2 is a block diagram of a medicament delivery system for controlled administration of medicament in accordance with one or more examples;

FIG. 3 shows a flowchart of method according to one or more embodiments of the disclosure; and

FIG. 4A and FIG. 4B illustrate a plurality of schematic representations of graphical user interfaces of a medicament delivery system according to one or more embodiments of the present disclosure.

### DETAILED DESCRIPTION

While the specification concludes with claims particularly pointing out and distinctly claiming what are regarded as embodiments of the present disclosure, various features and advantages may be more readily ascertained from the following description of example embodiments when read in conjunction with the accompanying drawings, in which:

Illustrations presented herein are not meant to be actual views of any particular automated medicament delivery device, insulin pump, component, or system, but are merely idealized representations that are employed to describe embodiments of the disclosure. Additionally, elements common between figures may retain the same numerical designation for convenience and clarity.

The following description provides specific details of embodiments. However, a person of ordinary skill in the art will understand that the embodiments of the disclosure may be practiced without employing many such specific details. Indeed, the embodiments of the disclosure may be practiced in conjunction with conventional techniques employed in the industry. In addition, the description provided below does not include all the elements that form a complete structure or assembly. Only those process acts and structures necessary to understand the embodiments of the disclosure are described in detail below. Additional conventional acts and structures may be used. The drawings accompanying the application are for illustrative purposes only and are thus not drawn to scale.

As used herein, the terms “comprising,” “including,” “containing,” “characterized by,” and grammatical equivalents thereof are inclusive or open-ended terms that do not exclude additional, unrecited elements or method steps, but also include the more restrictive terms “consisting of” and “consisting essentially of” and grammatical equivalents thereof.

As used herein, the singular forms following “a,” “an,” and “the” are intended to include the plural forms as well, unless the context clearly indicates otherwise.

As used herein, the term “may” with respect to a material, structure, feature, or method act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, the term “configured” refers to a size, shape, material composition, and arrangement of one or more of at least one structure and at least one apparatus facilitating operation of one or more of the structure and the apparatus in a predetermined way.

As used herein, the term “substantially” in reference to a given parameter, property, or condition means and includes to a degree that one skilled in the art would understand that the given parameter, property, or condition is met with a small degree of variance, such as within acceptable manufacturing tolerances. Via example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, etc.).

As used herein, the term “and/or” includes any and all combinations of one or more of the associated listed items.

Manual insulin deliveries are often utilized in conjunction with automated insulin delivery (AID) in insulin pumps with AID algorithms for controlling operation of the insulin pumps to maximize the glucose control outcomes of insulin pump users. However, while such AID systems and/or algorithms are typically designed to compensate for a wide range of variations in user bolus dosing behaviors, the AID algorithms are often specifically tailored to yield the most “time in range” (e.g., a percentage of time that a user's blood glucose levels remain in a specific target range) if the user's behaviors follow specific patterns that better compensate for the user's individual disturbances in glucose concentrations. Accordingly, if a user's behavior does not follow those specific patterns, the user may experience sub-optimal glucose control outcomes.

Target glucose concentration (i.e., target blood glucose level) is a relatively intuitive (e.g., easily understood) setting in AID systems that allows users (e.g., patients) to control the aggressiveness of the AID algorithms. Specifically, a target glucose concentration setting can be understood as an increase in medicament (e.g., insulin) delivery above basal levels if predicted glucose concentrations exceed a target glucose concentration, and a reduction or suspension of medicament (e.g., insulin) delivery if predicted glucose concentrations fall below the target glucose concentration. However, when utilizing AID systems, users may desire further adjustments to AID algorithm behaviors beyond a target glucose concentration. Nevertheless, including additional adjustable settings that users can individually set and adjust exacerbates existing complexities of AID devices, which can already present barriers to use for a significant portion of patients.

Embodiments of the present disclosure provide for an option to toggle (e.g., turn on and off) “additional adjustments” within the user's target glucose settings that reflect the user's preferences based on each setting. This option allows for further adjustment of AID system behaviors beyond the target glucose concentration. Specifically, in addition to setting the target glucose concentration, a user may select a user-selectable element or otherwise activate a setting that causes various AID algorithm input values to be automatically modified (e.g., adjusted) based on the target glucose concentration, such as penalization of glucose excursions, maximum insulin delivery limits, and various safety constraint settings. In view of the foregoing, enabling the user to optionally actuate or otherwise activate a setting to further adjust input values of the AID algorithm enables the user to toggle between levels of aggressiveness of the AID algorithm, while not having to adjust, adjust, or experiment with the intuitive target glucose concentration to change the levels of aggressiveness of the AID algorithm.

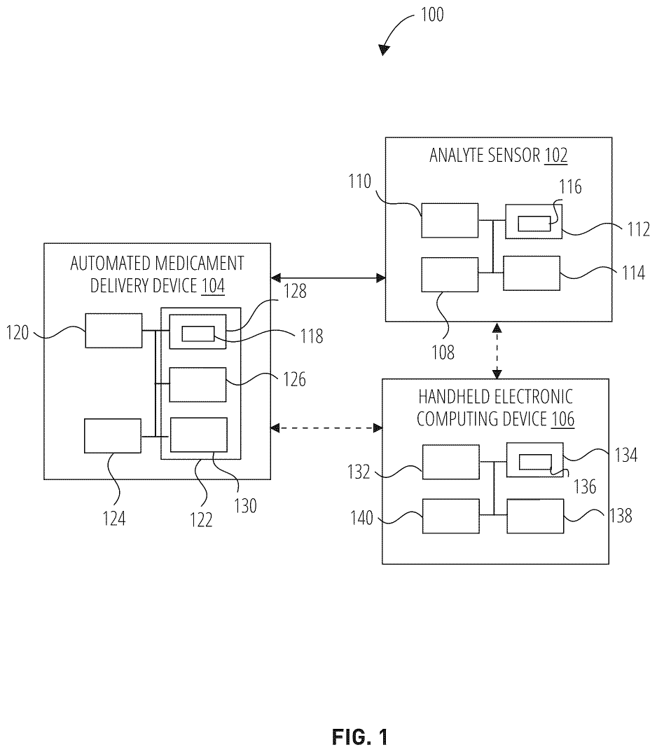

FIG. 1 is a schematic diagram showing a system 100 for administration of medicament to a user-body, in accordance with one or more examples.

In one or more examples, a system 100 may be capable of one or more modes of operation of administration of medicament (e.g., one or more distinct modes of operation, without limitation). Non-limiting examples of the one or more modes of operation include: fully automated administration of medicament, partially automated administration of medicament, and/or manual administration of medicament. In one or more examples, the system 100 may be capable of alternating between the multiple (e.g., two or more, without limitation) modes of operation. As a non-limiting example, the system 100 may alternate between one or more of: fully automated operation, partially automated operation, and/or manual operation.

The system 100 may administer medicament at least partially based on one or more values representative of amounts of one or more analytes present within a user-body (such values respectively an “analyte value”). The one or more analytes may include constituents of the user-body and foreign substances, such as medicaments, markers, metabolites, and combinations or sub combinations thereof, without limitation. Analyte values may include values representative of amounts of one or more analytes present within a user-body and values at least partially based on the same, such as, an A1C value, a blood glucose value (e.g., milligrams per decaliter (mg/dL), without limitation), an insulin-to-carbohydrate (I:C) ratio, or any combination thereof, without limitation.

The system 100 may also administer an amount of medicament at least partially based on user inputs (e.g., a user defined bolus amount or details related to a meal consumed or about to be consumed, such as number of carbohydrates, amount of fat, and amount of protein, without limitation). As used herein, administration of medicament responsive to a user input may be referred to as manual medicament delivery or manual delivery.

Non-limiting examples of medicaments administrable by system 100 include: insulin, glucagon-like peptide-1 receptor agonist (GLP-1), glucose-dependent insulinotropic polypeptide (GIP), or other hormones, insulin substitutes, and combinations of medicaments, such as two or more of insulin, GLP-1, and GIP, or other like hormones. While specific examples discussed herein may involve insulin or GLP-1, or GIP, this disclosure is not limited to those examples, and other medicaments do not exceed the scope. As a non-limiting example, glucagon, morphine, analgesics, fertility medicaments, blood pressure medicaments, chemotherapy drugs, arthritis drugs, weight loss drugs, without limitation are non-limiting examples of medicaments that are specifically contemplated. system 100.

The system 100 includes an analyte sensor 102 and an automated medicament delivery device 104. The system 100 may optionally include a handheld electronic computing device 106.

The analyte sensor 102 may be configured to obtain data related to one or more analytes within the user-body (“analyte data”). In various examples, the analyte data may include one or more analyte values. In various examples, the analyte sensor 102 is an analytical bio-sensing device, such as a continuous glucose monitor (CGM) or an integrated continuous glucose monitor (ICGM) (e.g., examples of commercially available analytical bio-sensing devices include the FREESTYLE LIBRE® 3 manufactured by Abbott or the DEXCOM® G7 manufactured by Dexcom, without limitation).

The analyte sensor 102 may include electrodes, enzymes, and/or a filament 108 and various electronic components. The filament 108 may be configured to obtain data related to one or more analytes within a user-body and provide the data to the various electronic components of the analyte sensor 102. The filament 108 may be configured to obtain the data directly from fluids of a user-body, including without limitation interstitial fluids of a user-body, from tissue of a user-body, combinations thereof, or in any other manner known in the art.

The analyte sensor 102 may include one or more processors 110, a memory 112, and communication equipment 114. The memory 112 may be coupled to the one or more processors 110. The memory 112 may be used for storing data, metadata, and programs for execution by the one or more processors 110. The memory 112 may include storage for storing data or instructions 116. The instructions 116 may include instructions for processing data obtained via the filament 108. When the instructions 116 are executed by the one or more processors 110, the instructions 116 cause the one or more processors 110 to process the data obtained via the filament 108. The instructions 118 may be implemented in hardware (e.g., one or more hardware processors of the one or more processors 110, such as an integrated circuit, application specific integrated circuit (ASIC), digital signal processor (DSP), or other logic circuit, without limitation), implemented in software (e.g., firmware, software, machine code, applications, without limitation), or a combination thereof. The instructions 116 for processing the data obtained via the filament 108 may include one or more instructions respectively for determining analyte values at least partially based on the data, or for sending the data, analyte values or both to the automated medicament delivery device 104 and/or the handheld electronic computing device 106.

The communication equipment 114 is configured to facilitate communication (e.g., a device or interface for wired communication, wireless communication, both wired and wireless communication, without limitation) of the analyte sensor 102 with other devices, including the automated medicament delivery device 104 and/or the handheld electronic computing device 106, without limitation. Such communication may be according to any appropriate wired or wireless communication protocol, such as WI-FI®, BLUETOOTH®, near-field communication (NFC), radio-frequency identification (RFID), or any other radio-frequency, infrared, or optical communication technology.

The automated medicament delivery device 104 may be configured to administer medicament to a user-body, such as subcutaneously into the user-body, without limitation, in accordance with one or more examples. In one or more examples, the automated medicament delivery device 104 may offer one or more modes of operation for administration of medicament to a user-body. When operating in some of the modes of operation, the automated medicament delivery device 104 may administer medicament at least partially responsive to analyte values, including without limitation analyte values received from analyte sensor 102. When operating in some further modes of operation, the automated medicament delivery device 104 may administer medicament at least partially responsive to user input. When operating some yet further modes of operation, the automated medicament delivery device 104 may administer medicament at least partially responsive to both analyte values and user input. Non-limiting examples of the one or more modes of operation offered by the automated medicament delivery device 104 include: fully automated administration of medicament, partially automated administration of medicament, or manual administration of medicament.

When operating in an operative mode that includes manual administration of medicament, the automated medicament delivery device 104 may administer medicament solely in response to a user input (e.g., delivers medicament in response to a user confirmation of delivery of medicament or in response to a user instruction to delivery medicament, without limitation). When operating in an operative mode that includes fully automated administration of medicament, the automated medicament delivery device 104 may administer medicament solely in response to analyte values (e.g., delivers medicament in response to one or more analyte values, without limitation). When operating in an operative mode that includes partially automated administration of medicament, the automated medicament delivery device 104 may administer medicament in response to analyte values and user input (e.g., delivers medicament in response to a user input and an analyte value, or alternately delivers medicament in response to a user input or in response to analyte values, without limitation).

Medicament administration may include administration of a basal amount of medicament regularly delivered a control interval (e.g., at a determined basal rate, without limitation) to keep analyte levels stale and within a determined or predetermined range. Medicament administration may also include administration of bolus amounts of medicament administered as an immediate bolus, an extended bolus, or a combination bolus (combination of an immediate bolus and an extended bolus). The bolus amount of medicament may be a correction bolus responsive to a change in analyte levels or a user defined bolus (e.g., responsive to user inputs provided, such as a user defined bolus amount or details related to a meal consumed or about to be consumed, such as number of carbohydrates, amount of fat, and amount of protein, without limitation).

The automated medicament delivery device 104 may include a delivery system 120, a controller 122 and a power source 124. The controller 122 may include one or more processors 126, a memory 128, and communication equipment 130. In one or more examples, the automated medicament delivery device 104, or portions thereof, may include a wearable device and may be secured to a user-body (e.g., secured via one or more adhesive layers attaching the automated medicament delivery device 104 to the skin of the user-body or a material that is secured to the user body, without limitation).

In various examples, the delivery system 120 is configured to cause an amount of medicament to move (e.g., flow, without limitation) toward and/or into a user-body.

In various examples, delivery system 120 may deliver amounts of medicament at least partially responsive to requests. In various examples, instructions 118 of the memory 128 may include instructions for determining and generating requests for delivery system 120. In various examples, instructions 118 may include instructions for determining one or more amounts of medicament, determining a timing for delivery of one or more amounts of medicament, and for generating one or more requests for delivery system 120 related to the same. When such instructions of instructions 118 are executed by one or more processors 126, the one or more processors 126 determine the amounts of medicament and timing of delivery, generate requests for the delivery system 120 at least partially based on the determined amounts and timing, and provide the requests to delivery system 120. In some embodiments, the requests and/or instructions for generating requests may be received from the handheld electronic computing device 106. Furthermore, activity (e.g., determined and generated requests for delivery system 120, administered doses, etc.) of the automated medicament delivery device 104 may be stored in the memory 128.

The communication equipment 130 is configured to facilitate communication (e.g., wireless communication, without limitation) of the automated medicament delivery device 104 with other devices, including without limitation communication between analyte sensor 102 and the automated medicament delivery device 104 and communication between the automated medicament delivery device 104 and the handheld electronic computing device 106.

The communication may be wired or wireless communication and may utilize any suitable communication protocol such as wireless networking protocol (e.g., Wi-Fi®, without limitation), a short-range wireless protocol (e.g., BLUETOOTH®, without limitation), a near-field communication standard, a cellular standard, or any other wireless optical or radio-frequency protocol. In various examples, the communication equipment 130 includes an Internet of Things (IOT) Subscriber Identity Module (SIM) card (e.g., a machine-to-machine SIM card, a Universal Integrated Circuit Card, without limitation).

The power source 124 is configured to supply power to the delivery system 120 and the various electronic components, such as the one or more processors 126, memory 128, communication equipment 130, etc. The power source 124 may be, as a non-limiting example, a power storage device (e.g., a battery, without limitation), a power inlet, a power regulator, or combination thereof.

In various examples, the handheld electronic computing device 106 is configured to communicate with the automated medicament delivery device 104 and the analyte sensor 102. The handheld electronic computing device 106 may be chosen from among a dedicated electronic device, a smart phone, a tablet computer, a wearable device (e.g., a smart watch, without limitation), a cloud computing device, and the like. As used herein, the term “handheld device” refers to a device sized and configured to be held/operated in one or more hands of a user.

The handheld electronic computing device 106 may include one or more processors 132, memory 134 that stores instructions 136 to be executed by the one or more processors 132, communication equipment 138, and a user interface 140. The one or more processors 132 and memory 134 may be configured/programmed to perform any of the operations discussed above, as well as other control operations for managing the automated medicament delivery device 104 and the analyte sensor 102.

The communication equipment 138 is configured to facilitate communication (e.g., wireless communication, without limitation) of the handheld electronic computing devices 106 with other devices, such as the automated medicament delivery device 104 and the analyte sensor 102. The communication may be wired or wireless communication, such as via a wireless networking protocol (e.g., Wi-Fi®, without limitation), a short-range wireless protocol (e.g., BLUETOOTH®, without limitation), a near-field communication standard, a cellular standard, or any other wireless optical or radio-frequency protocol. In some of these examples, the automated medicament delivery device 104 and the handheld electronic computing devices 106 are paired via the short-range wireless protocol (e.g., paired via BLUETOOTH®, without limitation) and successful message transmissions between the automated medicament delivery device 104 and the handheld electronic computing devices 106 may be acknowledged.

The user interface 140 is configured to provide a user with information and obtain information from the user via one or more of a display, an audio speaker, an LED, a vibration motor, a button (e.g., a mechanical button, capacitive button, without limitation), a gesture-based interface, and the like.

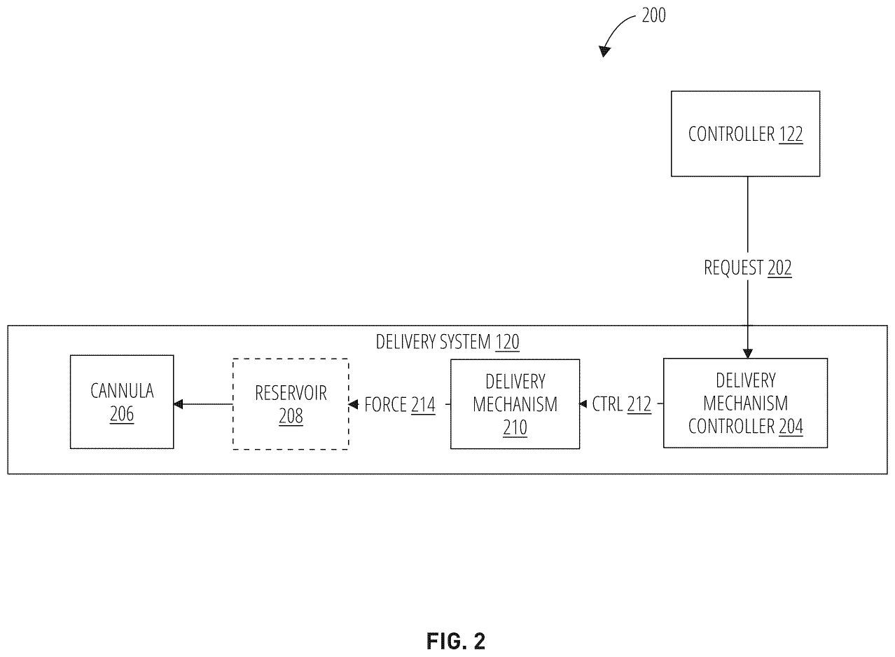

FIG. 2 is a block diagram of a medicament delivery system 200 for controlled administration of medicament to a user-body, in accordance with one or more examples. In some embodiments, the medicament delivery system 200 may include or form a portion of the automated medicament delivery device 104 of FIG. 1.

The controller 122 is configured to manage the automated medicament delivery device 104 and, more generally, administration of medicament to a user-body. In one or more examples, controller 122 may be implemented by instructions 118 and one or more processors 126 of automated medicament delivery device 104 of FIG. 1. Furthermore, activity (e.g., determined and generated requests for delivery system 120, administered doses, etc.) of the automated medicament delivery device 104 and/or the medicament delivery system 200 may be stored in a memory 112.

In various examples, the controller 122 and the delivery system 120 may be realized in different devices (e.g., controller 122 may be realized in a physically different device (or devices) than the delivery system 120 is realized, such as the handheld electronic computing device 106, without limitation), or in the same device. When realized in different devices, functionality of the controller 122 and the delivery system 120 may be implemented, at least in part, by respective memory and one or more processors of their respective devices. When realized in a same device, functionality of the controller 122 and the delivery system 120 may be implemented, at least in part, by like memory and like one or more processors, respective memory and respective one or more processors, or a combination thereof. Non-limiting examples of devices in which the controller 122, or a portion thereof, may be realized include: a handheld electronic computing device, such as a dedicated electronic device, a smart phone, a tablet computer, a wearable device (e.g., a smart watch, without limitation), a cloud computing device, and the like.

In various examples, the controller 122 may be configured to receive analyte data (e.g., from the analyte sensor 102, without limitation) including analyte values. In one or more examples, the controller 122 may determine information about analytes within a user-body at least partially based on analyte data, for example, amounts, trends, distributions, without limitation. The controller 122 may analyze information about analytes in a user-body and may present the information and/or analysis to a patient, caregiver, or healthcare provider, as a non-limiting example, via an application (e.g., executing on a personal computer, a smart phone, a cloud server, and/or combinations thereof).

In various examples, the controller 122 may be configured to receive information from inputs from the patient or a caregiver (e.g., when the patient ate a meal or when the patient exercised, without limitation), and inputs from other electronic devices (e.g., information from a smartwatch, without limitation) and to utilize such information as discussed herein. For example, in various examples, the controller 122 may utilize some or a totality of such information to determine amounts of medicament to administer and timing of administration of medicament. Further, controller 122 may also be configured to determine requests, including requests 202 to administer doses, and send those requests to the automated medicament delivery device 104.

In various examples, the controller 122 may be configured to determine a target dose amount to administer to a user of the medicament delivery system 200. The controller 122 may determine a target dose amount at least partially based on therapy parameters, meal information, analyte values, and a control algorithm, without limitation.

In the context of insulin therapy to treat diabetes, therapy parameters may include insulin sensitivity factor (ISF), carbohydrate ratio (CR), amount of daily dose of long-acting insulin (LAI), a current glucose value, and derivatives thereof without limitation. The timing and target dose amounts associated with requests generated by controller 122 may be governed by one or more control algorithms, discussed below. For instance, the timing and target dose amounts associated with requests generated by controller 122 may be governed by one or more AID algorithms.

The controller 122 may send a request 202 to administer a dose to delivery system 120, and more specifically, delivery mechanism controller 204. The request 202 to administer a dose may include the target dose amount determined by the controller 122.

The cannula 206 is insertable into a user-body (e.g., with a tip thereof positioned subcutaneously, without limitation) and is configured to provide medicament to a user-body (e.g., subcutaneously into the user-body, without limitation).

The reservoir 208 is configured to store and retain a medicament therein. As a non-limiting example, the reservoir 208 may be a hollow body, a chamber, a vial, without limitation. In various examples, the reservoir 208 is a fluid reservoir for holding medicament and may be, as a non-limiting example, formed from the walls of a cartridge. In the cartridge example, the delivery system 120 may include a chamber (i.e., a space or region defined within the delivery system 120) configured to receive and hold a prefilled (prefilled with medicament) cartridge, eject an exhausted cartridge, and optionally receive a prefilled cartridge to replace (i.e., a replacement cartridge) the exhausted cartridge. Generally speaking, a volume of fluid in the reservoir 208 will be greater in a pre-filled state than the volume in an exhausted state. Additionally or alternatively to the cartridge example, the delivery system 120 is a multi-part delivery device where one of the two parts includes the reservoir 208 and the other one of the two parts includes the delivery mechanism controller 204. The other one of the two parts may optionally further include the controller 122. Either one of the two parts may optionally include the delivery mechanism 210 (e.g., a pump mechanism, without limitation). The one of the two parts that includes the reservoir 208 is disposable (i.e., a “disposable part”) and configured to be removable secured to the other part of the medicament delivery system 200. When the reservoir 208 is exhausted, the disposable part may be removed and a replacement part including a reservoir 208 optionally in a pre-filled state.

The delivery mechanism 210 is configured to urge fluid in the reservoir 208 toward an interface for dispensing fluid (interface not shown). In various examples, the delivery mechanism 210 may be positioned adjacent to the reservoir 208. The delivery mechanism 210 is configured to cause an amount of the medicament to be administered to the user-body by causing the amount to flow from the reservoir 208 toward and into a user-body via cannula 206, which is in fluidic communication with the reservoir 208. In various examples, the delivery mechanism 210 may utilize any suitable mechanism to generate positive displacement or negative displacement to transfer amounts of medicament from the reservoir 208 toward the cannula 206 and a user-body. Non-limiting examples of mechanisms include a ratchet gear pump, a peristaltic pump, a linear peristaltic pump, a piston pump, a gear pump, a bellows pump, or a diaphragm pump.

For example, the delivery mechanism 210 may apply a force to an urging mechanism (e.g., a plunger, flexible-walled tube, without limitation) free to move within the reservoir 208, and via such a force, move the urging mechanism in a direction that urges fluid in the reservoir 208 toward the aforementioned interface. In one or more examples, the delivery mechanism 210 may include an electrical motor (e.g., an AC or DC motor) that produces a force to, directly or indirectly, move the urging mechanism to perform a delivery action. A delivery action dispenses at a predetermined rate (i.e., a predictable amount of fluid over a predictable duration of time). The delivery mechanism 210 may be capable of multiple rates of delivery, and in one or more examples, may be preconfigured to use a same rate of delivery all the time, or, in some cases, may be provided discretion to determine a rate of delivery consistent with a target dose amount included with a request 202.

Such an electric motor may be a current controlled electric motor, voltage controlled electric motor, pulse-width controlled electric motor, or combination or sub combination thereof. Such an electronic motor may be directly or indirectly digitally controlled. A control signal 212 may be determined and generated by the delivery mechanism controller 204 to correspond to a delivery action. The control signal 212 may also be referred to herein as a “command 212” or an “instruction 212.”

The delivery mechanism controller 204 may generate control signals 212 corresponding to one or more delivery actions at least partially based on a request 202 to administer a dose received from the controller 122 or elsewhere (e.g., the handheld electronic computing device 106). The control signal 212 may include first control signals to cause the delivery mechanism 210 to generate a resultant force 214, and a second, different control signal to cause delivery mechanism 210 to not or stop generating the force 214. Utilizing control signals 212, the delivery mechanism controller 204 may control a length of a duration of time that the delivery mechanism 210 produces the force 214 and applies it to dispense fluid from the reservoir 208, and indirectly, an amount of fluid dispensed from the reservoir 208.

When delivery mechanism controller 204 generates the control signal 212 in response to a request 202 to administer a dose from the controller 122 or elsewhere, it may generate the control signal 212 at least partially based on a value of a target dose amount included with, or indicated by, the request 202 to administer a dose. One or more delivery actions may be utilized to dispense an amount fluid corresponding to a dose amount determined by the controller 122. For example, a fluid amount dispensed according to a delivery action may be less than a dose amount. Generally speaking, the delivery mechanism 210, and the delivery system 120, are agnostic to the purpose for which fluid is dispensed and unaware of what constitutes a working amount of fluid to administer a dose, or series of doses, of medicament. So, while it may be desirable that a fluid amount dispensed according to one or more delivery actions will be exactly the same as a target dose amount, some negligible difference is specifically contemplated, and what is considered “negligible” will depend on specific operation conditions.

In one or more examples, the delivery mechanism controller 204 may be configured to determine and generate feedback information about delivery actions, such as times of delivery actions and dispensed amounts, without limitation. Feedback information may be generated based on information generated by the delivery mechanism 210 or by sensors utilized by the delivery mechanism controller 204 to monitor operation of the delivery mechanism 210 (sensors not depicted). For example, sensors to monitor mechanical movement, current consumption, a voltage profile of an electric motor, without limitation. Such information may be logged and provided to and stored at the controller 122 and/or the handheld electronic computing device 106, without limitation, for e.g., later processing or reading, without limitation. For example, the logs can be processed to determine patterns that may be utilized to determine whether the delivery system 120 is operating as expected (e.g., in a predictable manner, without limitation), and if a difference between actual and expected operation exceeds a threshold, the delivery mechanism controller 204 may be updated (e.g., firmware, parameters, or both, of the delivery mechanism controller 204 may be updated, without limitation) to compensate or correct for the difference. Additionally or alternatively to updating the firmware or parameters, in a multi-part system, one or more parts including the delivery mechanism controller 204 or the delivery mechanism controller 204 may be indicated as needing replacement (e.g., an alarm or alert is generated at the delivery system 120, the medicament delivery system 200, a mobile device, and/or computer in communication therewith, without limitation).

### Control Algorithm Architecture

As noted above, values of target dose amounts and timing of requests to administer the target dose amounts generated by the controller 122 may be governed by one or more control algorithms (AID algorithms) implemented at the controller 122. Generally speaking, such a control algorithm may, via one or more control actions, try to cause an amount of analyte in the body (represented by values captured by, or at least partially based on, an analyte sensor or monitor, without limitation) to track (e.g., at least substantially match) a target amount of analyte (in control terms, the target amount of analyte is the “set point”) in the body. The control actions may include an amount and a timing of administration of doses of medicament that functions as a therapeutic agent in the body.

In one or more examples, a control algorithm may employ a modular design in which core functionality may be separated from dependent functionality. Dependent functionality includes, as non-limiting examples, functionality that may be implementation-specific to a current environment, such as software abstraction for an analyte sensor. Such dependent functionality may include software services which interface with implementation-specific features that affect inputs or outputs to the control algorithm. Dependent functionality may include, as a non-limiting example, functionality for managing algorithm initialization and upload of administration history, managing the control algorithm's state and data therapy variables, and maintaining cycle-to-cycle data utilized by the algorithm such as analyte values, current or historical. Dependent functionality may include functionality responsible for sending requests to administer doses to delivery system 120 which are determined by the control algorithm.

Transmission of data, including without limitation, a request 202 to administer a dose, may occur over wired, wireless, or a combination thereof communication paths, in a synchronous or asynchronous manner. In one or more examples, the control algorithm may include one or more layers to provide safety or other operational constraints (e.g., for edge case handling, without limitation).

In one or more examples, a control algorithm may determine a target dose amount to include within a request at least partially based on a dynamic model of a user-body's response, in terms of amount of analyte in the user-body, to administration of analyte to the user-body. The control algorithm may determine a future amount of analyte or a change in amount of analyte over a predetermined duration of time for a respective dose amount and compare the determined future amount or change to a target amount or change. The control algorithm may determine target dose amounts according to control intervals that occur according to a predetermined schedule, on-demand, or both. In one or more examples, the control intervals may correspond to diurnal intervals such as day-night, weeks, days, twenty-four (24) hours, single hours, and sub-intervals of the same, such as 5-minute intervals.

In some cases, the control algorithm may be or include a control algorithm that handles constraints, such as a model-predictive-control (MPC) algorithm. Non-limiting examples of constraints include: upper and lower bounds on analyte levels can be set to prevent dangerous hypo- or hyperglycemia; medicament delivery rates capable by delivery system 120 can be constrained to prevent over- or under-dosing; and considerations related to medicament-on-board to, e.g., prevent stacking of medicament doses waiting to work on analyte in the body (e.g., stacking of insulin waiting to work on glucose, without limitation).

One or more examples discussed herein may refer to administering medicament or a medicament therapy to a user or the user-body. Such discussion is intended to encompass examples where medicament or a medicament therapy is administered to a user by automated medicament delivery devices discussed herein, examples where requests to administer doses in accordance with administering medicament or medicament therapy to the user or user-body are generated by a controller and sent to a delivery device, and examples where instructions (e.g., control signals, without limitation) in accordance with dose amounts and timing included with such requests to administer doses are generated by a delivery mechanism controller and sent to a delivery mechanism.

As noted above, target glucose concentration (i.e., target blood glucose level) is a relatively intuitive (e.g., easily understood) setting in AID systems that allows users (e.g., patients) to control the aggressiveness of the AID algorithms, and a result, their medicament administration and regime. Specifically, a target glucose concentration setting can be understood as an increase in medicament (e.g., insulin) delivery above basal levels if predicted glucose concentrations exceed a target glucose concentration, and a reduction or suspension of medicament (e.g., insulin) delivery if predicted glucose concentrations fall below the target glucose concentration. However, when utilizing AID systems, users may desire further adjustments to AID algorithm behaviors beyond a target glucose concentration. Nevertheless, including additional adjustable settings that users can individually set and adjust exacerbates existing complexities of AID devices, which can already present barriers to use for a significant portion of patients.

As mentioned briefly above, embodiments of the present disclosure provide for systems and methods that provide an option to toggle (e.g., turn on and off) “additional adjustments” within a user's target therapy parameter settings that reflect the user's preferences based on each setting. This option allows for further adjustment of AID algorithm and AID system behaviors beyond the target therapy parameters (e.g., glucose concentration). Specifically, in addition to setting the target therapy parameter, a user may select a user-selectable element or otherwise activate a setting that causes various AID algorithm input values to be automatically modified (e.g., adjusted) based on the target therapy parameter. Those various AID algorithm input values may include values representing penalization of glucose excursions, maximum insulin delivery limits, and various safety constraint settings. In view of the foregoing, enabling the user to optionally actuate or otherwise activate a setting to further adjust input values of the AID algorithm enables the user to toggle between levels of aggressiveness of the AID algorithm, while not having to adjust, adjust, or experiment with the intuitive target glucose concentration to change the levels of aggressiveness of the AID algorithm.

As used herein, the term “aggressiveness” when used in reference to an AID algorithm refers to how assertively (e.g., how promptly and significantly) the AID algorithm adjusts medicament (e.g., insulin) delivery to maintain blood glucose levels within a target range. A more aggressive AID algorithm will make larger and/or more frequent adjustments to medicament delivery in response to changes in blood glucose levels, aiming to more quickly correct deviations from the target range. A more aggressive AID algorithm maintains tighter glucose control but may also increase the risk of hypoglycemia if not carefully managed. Conversely, a less aggressive algorithm will make smaller and/or less frequent adjustments, which might result in more stable medicament delivery but may allow for larger fluctuations in blood glucose levels.

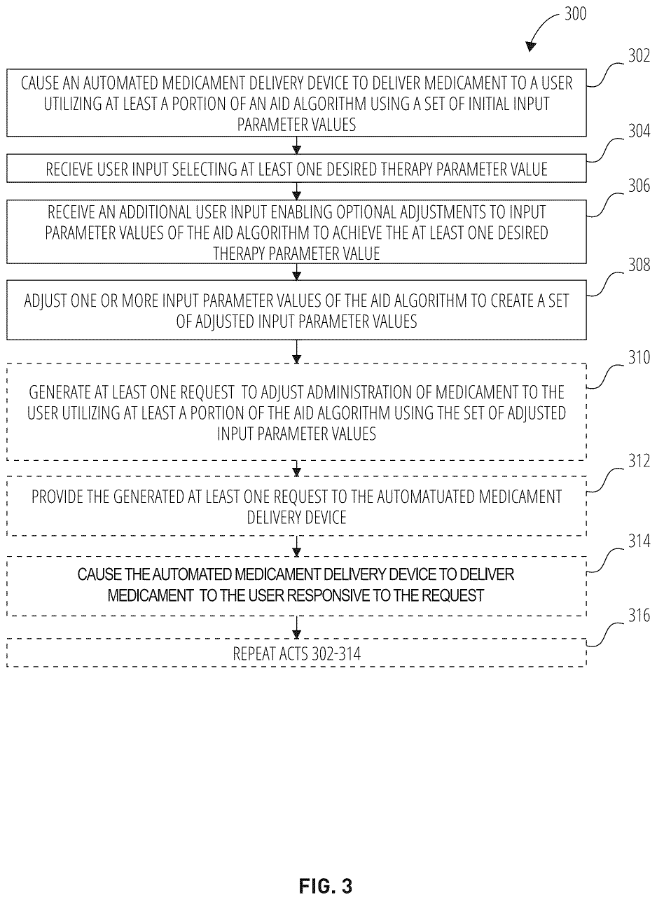

FIG. 3 shows a flowchart of a method 300 of managing medicament therapy for one or more users. In some embodiments, one or more acts of the method 300 may be performed and/or executed by the controller 122 of the automated medicament delivery device 104 and/or the medicament delivery system 200. However, the disclosure is not so limited, and one or more acts of the method 300 may be performed, executed, and/or initiated by the delivery mechanism controller 204 of the delivery system 120, other controllers of the system 100, the handheld electronic computing device 106, and/or one or more other remote devices. For purposes of the present disclosure, the acts of the method 300 are described as being performed, executed, and/or initiated by the controller 122 of the automated medicament delivery device 104.

The method 300 may include causing the automated medicament delivery device 104 to deliver medicament to a user utilizing at least a portion of an AID algorithm having (e.g., using, inputting, incorporating, etc.) a set of initial input parameter values, as shown in act 302 of FIG. 3. For instance, the controller 122 of the automated medicament delivery device 104 may cause the automated medicament delivery device 104 to deliver medicament to the user utilizing at least a portion of the AID algorithm having (e.g., using, inputting, incorporating, etc.) the set of initial input parameter values. As noted above, the AID algorithm may govern delivery of the medicament and may utilize the initial input parameter values to determine and administer medicament therapy.

For instance, the method 300 may include causing the automated medicament delivery device 104 to deliver medicament to the user's body via any of the manners described above in regard to FIG. 1 and FIG. 2. Put another way, the method 300 may include controlling operation of the automated medicament delivery device 104 and causing the automated medicament delivery device 104 to deliver medicament utilizing at least a portion of an AID algorithm.

In some embodiments, the initial input parameter values may be input by the user (e.g., patient), automatically generated based at least partially on received data (e.g., analyte data, exercise data, and/or meal data), and/or input by a caregiver (e.g., healthcare provider) according to a diabetes treatment plan or other drug delivery regimen. In one or more embodiments, the initial input parameter values may include values for one or more of a Q:R Ratio, a TDI (Total Daily Insulin) value, an insulin-on-board (IOB) constraint duration of insulin action value, an insulin sensitivity factor (ISF) value, a carbohydrate ratio (CR), an amount of daily dose of long-acting insulin (LAI), amounts of anticipated bolus doses, recommended bolus dose timings, carb count of meals, a basal rate, a basal rate profile, a target blood glucose level, periods of exercise, changes in insulin sensitivities, or glucose concentrations when a bolus may be automatically adjusted. The set of initial input parameter values may cause the AID algorithm to exhibit a first level of aggressiveness. In some embodiments, the set of initial input parameter values may be stored within one or more of the memory 128 of the automated medicament delivery device 104, the memory 134 of the handheld electronic computing device 106, and/or a database of a remote device (e.g., a server).

In the embodiments described herein, the Q:R Ratio comprises a parameters that may be utilized by the AID algorithm to determine an aggressiveness of the AID algorithm. For example, values of Q and R or the Q:R Ratio itself may be utilized in a cost function to optimize a balance between achieving desired blood glucose levels and minimizing the risk of hypoglycemia and/or other side effects. In exemplary embodiments parameters of the cost function, such as the values of Q and R or the Q:R Ratio, may be adjusted based on a user's target glucose value setting to increase or decrease aggressiveness of drug delivery. An exemplary cost function for determining a cost J is:

$$J ( k ) = Q {\sum }_{i = k + 1}^{P} {( G ( i ) - S P ( i ) )}^{2} + R {\sum }_{i = k + 1}^{C} {( I ( i ) - b ( i ) )}^{2} \qquad ( Equation 1 )$$

where J(k) is the cost of a specified insulin dose for cycle k;

$$Q {\sum }_{i = k + 1}^{P} {( G ( i ) - S P ( i ) )}^{2}$$

is the weighted glucose cost component; and

$$R {\sum }_{i = k + 1}^{C} {( I ( i ) - b ( i ) )}^{2}$$

is the weighted insulin cost component. Q is a weight coefficient for the glucose cost component. The glucose cost component represents the weighted sum of the deviations squared in the glucose level of the user (G(i)) over a future time horizon (cycles k+1 to P) relative to a target glucose level (or setpoint) SP(i) if the specified basal insulin dose is delivered. R is a weight coefficient for the insulin cost component. The insulin cost component represents the costs of the squares of the deviations in insulin delivery amounts (I(i)) delivered over a time period (cycles k+1 to C) in the future relative to a pre-set basal insulin dose (b(i)). The total cost J(k) is the sum of the weighted glucose cost and the weighted insulin cost for a particular cycle k. A cycle has a fixed interval, such 5 minutes. This formulation of the cost function is one of many possible formulations that penalize the deviations, and other formulations, such as a linear formulation (sum of the absolute values of deviations), asymmetric formulations, and other forms are also possible to be implemented.

The controller 122 and AID algorithm attempt to minimize the aggregate penalty of the cost function over a wide range of possible dosages and resulting projected glucose concentrations. The cost function is applied to the possible dosages, and the dosage with the best cost function value is selected. Depending on how the cost function is configured, the best value may be the lowest value or the highest value. In exemplary embodiments, the best value may be the lowest value of the total cost J. Using the dosage with the best cost function value, a request 202 or control signal 212 based on that dosage may be generated by the controller 122 and sent to the delivery system 120 to cause the delivery system 120 to deliver the determined insulin dose to the user.

Conventionally, coefficients such as Q and R in a cost function are not adjusted based on a user's target blood glucose value. Instead, the glucose cost component itself is impacted because it takes into account the user's target blood glucose value (or setpoint) SP(i). Adjusting weight coefficients Q and/or R and/or the Q:R Ratio is an additional way to impact the cost function and the aggressiveness of an AID algorithm.

Q represents an importance of maintaining blood glucose levels within a target range. Higher values of Q place more emphasis on keeping blood glucose levels close to a desired target, while penalizing deviations more heavily (e.g., while responding to deviations in blood glucose levels more aggressively with medicament administration). Conversely, R represents a cost and/or a risk associated with administering medicament (e.g., insulin). Higher values of R penalize the use of medicament more heavily, while encouraging use of a minimum effective dose to achieve a desired blood glucose levels. In view of the foregoing, a reduction of the Q:R ratio results in an increase in the aggressiveness of the AID algorithm. Adjusting the values of Q and/or R can create a personalized insulin therapy plan that balances the need for tight glucose control with the risk of hypoglycemia and other complications. Accordingly, adjusting the values of Q and/or R can help in fine-tuning medicament doses to achieve optimal glycemic control while minimizing adverse effects.

The method 300 may further include receiving user input selecting at least one desired therapy parameter value, as shown in act 304 of FIG. 3. For example, the controller 122 of the automated medicament delivery device 104 may receive the user input selecting at least one desired therapy parameter value. In some embodiments, the user input may be received via the user interface 140 of the handheld electronic computing device 106 and then communicated to the automated medicament delivery device 104 via any of the manners described above in regard to FIG. 1 and FIG. 2. In additional embodiments, the user input may be received via a user interface of the automated medicament delivery device 104 or any other device.

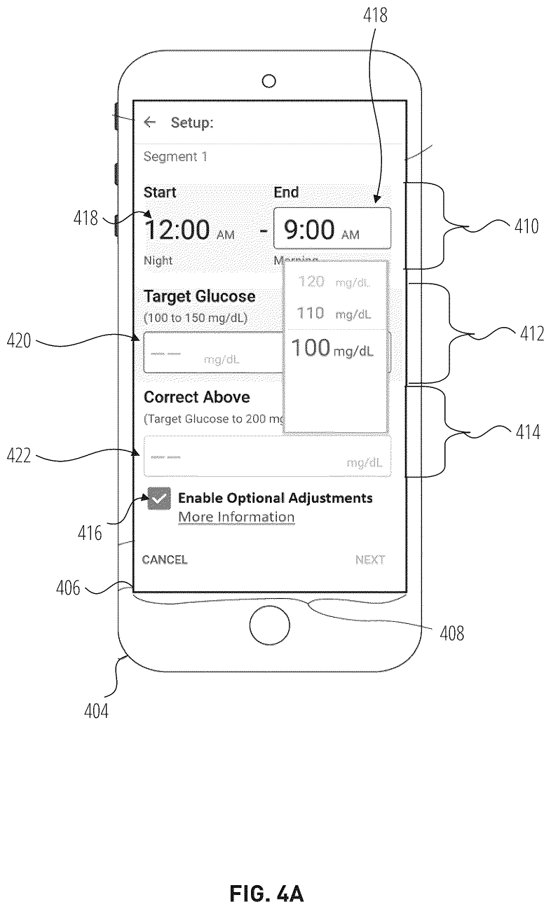

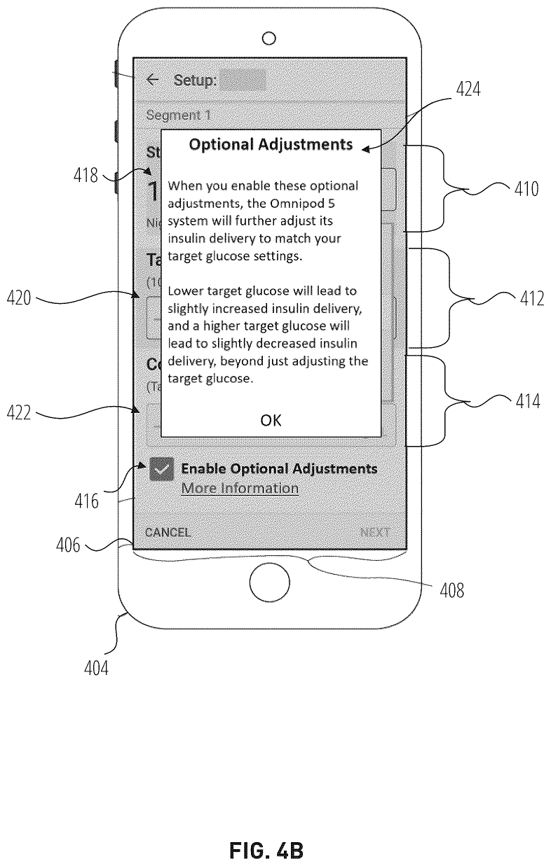

In some embodiments, the user input may include on or more of an interaction with a display (e.g., touchscreen), an audio speaker, a button (e.g., a mechanical button, capacitive button, without limitation), or a gesture-based interface of the user interface 140 (FIG. 1) of the handheld electronic computing device 106 (FIG. 1). As is described in greater detail in regard to FIG. 4A and FIG. 4B, in one or more embodiments, the user input may include a selection of at least one desired therapy parameter value within a graphical user interface (GUI).

In one or more embodiments, the at least one desired therapy parameter value may include a target blood glucose level. As noted above, a target blood glucose level may be a relatively intuitive parameter (e.g., setting) that a user can select to control an aggressiveness of an AID algorithm. In additional embodiments, the at least one desired therapy parameter value may include a target pre-prandial glucose level, a TDI (Total Daily Insulin) value, a basal rate for a specific period of time, a daily basal rate, a pre-defined period of exercise, other differing insulin sensitivities for a specific period of time, or a daily change in insulin sensitivity.

The method 300 may further include, responsive to receiving the user input selecting at least one desired therapy parameter value, receiving an additional user input enabling optional adjustments to input parameter values of the AID algorithm to achieve the at least one desired therapy parameter value, as shown in act 306 of FIG. 3. For example, the controller 122 of the automated medicament delivery device 104 may receive the additional user input selecting “enabling optional adjustments,” or similar language, to input parameter values of the AID algorithm to achieve the at least one desired therapy parameter value. In some embodiments, the additional user input may be received via the user interface 140 of the handheld electronic computing device 106 and then communicated to the automated medicament delivery device 104 via any of the manners described above in regard to FIG. 1 and FIG. 2. In additional embodiments, the additional user input may be received via a user interface of the automated medicament delivery device 104.

In some embodiments, the additional user input may include on or more of an interaction with a display (e.g., touchscreen), an audio speaker, a button (e.g., a mechanical button, capacitive button, without limitation), or a gesture-based interface of the user interface 140 of the handheld electronic computing device 106. As is described in greater detail in regard to FIG. 4A and FIG. 4B, in one or more embodiments, the additional user input may include a selection of user-selectable element (e.g., a toggle switch) within a graphical user interface (GUI) that enables or disables the optional adjustments to input parameter values of the AID algorithm.

The method 300 further includes, responsive to receiving the additional user input enabling the optional adjustments to input parameters values of the AID Algorithm, adjusting one or more of the initial input parameter values of the AID algorithm to create a set of adjusted input parameter values, as shown in act 308 of FIG. 3. For example, the controller 122 of the automated medicament delivery device 104 may adjust one or more of the initial input parameter values of the AID algorithm to create a set of adjusted input parameter values. In additional embodiments, one or more of the handheld electronic computing device 106 or a remote device may adjust one or more of the initial input parameter values of the AID algorithm to create the set of adjusted input parameter values. In some embodiments, adjusting one or more of the initial input parameter values of the AID algorithm to create a set of adjusted input parameter values includes automatically adjusting one or more of the initial input parameter values of the AID algorithm based at least partially on the selected at least one desired therapy parameter value and without additional user input.

When utilized by the AID algorithm, the set of adjusted input parameter values may cause the AID algorithm to exhibit a second, different level of aggressiveness. In some embodiments, the second, different level of aggressiveness is an increased level of aggressiveness relative to the first level of aggressiveness. In other embodiments, the second, different level of aggressiveness is a decreased level of aggressiveness relative to the first level of aggressiveness. In some embodiments, the set of adjusted input parameter values may be stored within one or more of the memory 128 of the automated medicament delivery device 104, the memory 134 of the handheld electronic computing device 106, and/or a database of a remote device (e.g., a server).

In some embodiments, adjusting one or more of the initial input parameter values of the AID algorithm may include adjusting one or more initial input parameter values, such as, for example, values of coefficients Q and/or R, values of a Q:R Ratio, a TDI (Total Daily Insulin) value, an insulin-on-board (IOB) constraint duration of insulin action value, an insulin sensitivity factor (ISF) value, a carbohydrate ratio (CR), an amount of daily dose of long-acting insulin (LAI), amounts of anticipated bolus doses, carb count of meals, a basal rate, pre-defined periods of exercise, or other periods of changed insulin sensitivities. In one or more embodiments, adjusting one or more of the initial input parameter values of the AID algorithm may include adjusting each of a Q:R Ratio, a TDI (Total Daily Insulin), and an insulin-on-board (IOB) constraint duration of insulin action value.

In some embodiments, the one or more of the initial input parameter values of the AID algorithm may be adjusted based at least partially on the at least one desired therapy parameter value and current therapy data. The current therapy data may include one or more of current (e.g., measured) CGM data (e.g., current blood glucose levels), insulin-on-board (IOB) data, a TDI (Total Daily Insulin) value, carb count of meals, a current basal rate, anticipated meals, a currently carbohydrate ratio (CR), an amount of daily dose of long-acting insulin (LAI), amounts of anticipated bolus doses, and recommended bolus dose timings. For instance, in some embodiments, the one or more of the initial input parameter values of the AID algorithm may be adjusted based at least partially on based on differences between the at least one desired therapy parameter value and the current therapy data (e.g., a difference between a current blood glucose level and the desired or target blood glucose level).

As a non-limiting example, in embodiments where the at least one desired therapy parameter value is a desired (e.g., target) blood glucose level, input parameter values (e.g., the initial input parameter values) of the AID algorithm may be adjusted as shown below in Table 1 to form the set of adjusted input parameter values.

TABLE 1

| | Optional adjustments that may | | |
|---|---|---|---|
| | be activated with each target glucose setting | | |
| User's target glucose | | | IOB Constraint Duration |
| value setting | Q:R Ratio | Input TDI | of Insulin Action |
| 150 | 27 | −10% | 4 |
| 140 | 23 | −7.5% | 3.75 |
| 130 | 28 | −5% | 3.5 |
| 120 | 13 | −2.5% | 3.25 |
| 110 | 7 | 0% | 3 |
| 100 | 4 | +5% | 2.5 |

Responsive to creating the set of adjusted input parameter values, the method 300 may optionally include generating at least one request 202 to adjust administration of medicament to the user (e.g., adjust operation of the automated medicament delivery device 104) utilizing at least a portion of the AID algorithm having (e.g., using, inputting, incorporating, etc.) the set of adjusted input parameter values, as shown in act 310 of FIG. 3. In one or more embodiments, the controller 122 may generate the at least one request 202. In additional embodiments, the request 202 may be generated by one or more of the handheld electronic computing device 106 or a remote device (e.g., a server) and communicated to the automated medicament delivery device 104 (discussed below). In some embodiments, the request 202 may include instructions to administer basal dosing and/or a bolus dose determined by utilizing at least a portion of the AID algorithm having (e.g., using, inputting, incorporating, etc.) the set of adjusted input parameter values.

Additionally, the method 300 may include optionally providing the generated at least one request to the automated medicament delivery device 104, as shown in act 312 of FIG. 3. For instance, the controller 122 of the automated medicament delivery device 104 may provide the at least one request 202 to the delivery mechanism controller 204 of the delivery system 120 of the automated medicament delivery device 104 via any of the manners described above in regard to FIG. 1 and FIG. 2.

Moreover, the method 300 may optionally include causing the automated medicament delivery device 104 to deliver insulin to the user's body in accordance with the request 202, as shown in act 314 of FIG. 3. For instance, the method 300 may include causing the medicament delivery system 200 to deliver insulin to the user's body responsive to the request 202 via any of the manners described above in regard to FIG. 1 and FIG. 2. Put another way, the method 300 may include adjusting operation of the medicament delivery system 200 of the automated medicament delivery device 104 and causing the medicament delivery system 200 of the automated medicament delivery device 104 to deliver insulin according to the adjusted operation.

Furthermore, the method 300 may optionally include iteratively repeating act 302 through act 314, as shown in act 316 of FIG. 3. For example, the controller 122 may iteratively repeat act 302 through act 316. In at least some iterations, a respective set of initial input parameter values may represent a previous set of adjusted input parameter values from an immediately preceding iteration.

Repeating act 302 through act 314 may enable the automated medicament delivery device 104 and an associated AID algorithm to exhibit a level of aggressiveness desired by a user and to adjust an exhibited level of aggressiveness without requiring the user to adjust individual settings beyond an initial therapy parameter (e.g., a target blood glucose level). As a result of the foregoing, the automated medicament delivery device 104 of the present disclosure may provide relatively easy, simple, and intuitive user experience, while empowering the user to personalize their therapy regime.

FIG. 4A and FIG. 4B illustrate a collection of user interfaces including features of the automated medicament delivery device 104 (FIG. 1) and the handheld electronic computing device 106 of the system 100 according to one or more embodiments of the present disclosure. In particular, referring to FIG. 1, FIG. 4A, and FIG. 4B together, the user interfaces show features of the automated medicament delivery device 104 and/or the handheld electronic computing device 106 of the system 100. As will be described in more detail below, the automated medicament delivery device 104 and/or the handheld electronic computing device 106 as described in regard to FIG. 1 through FIG. 3 can provide, along, and/or in combination with the other components, one or more graphical user interfaces (“GUIs”). In particular, the automated medicament delivery device 104 and/or the handheld electronic computing device 106 may allow a user to interact with a collection of display elements for a variety of purposes, including selecting at least one desired therapy parameter value and enabling optional adjustments to input parameters of an AID algorithm. For instance, FIG. 4A and FIG. 4B and the description that follows illustrate various example embodiments of the user interfaces and features that are in accordance with one or more embodiments of the present disclosure.

For example, FIG. 4A illustrates a user device 404 that may implement one or more of the components or features of the automated medicament delivery device 104 and/or the handheld electronic computing device 106. For purposes of the present disclosure, the user device 404 may include the handheld electronic computing device 106 and/or a portion of the automated medicament delivery device 104. In the example shown in FIG. 4A, the user device 404 is a handheld device, such as a tablet device. In additional or alternative examples, however, any other suitable computing device, such as, but not limited to, a mobile phone device, larger wireless device, laptop or desktop computer, a personal digital assistant device, and/or any other suitable computing device can perform one or more of the processes and/or operations described herein.

The user device 404 includes a touch screen display 406 that can display user interfaces. Furthermore, the user device 404 receives and/or detects user input via the touch screen display 406. As used herein, a “touch screen display” refers to the display of a touch screen device. In one or more embodiments, a touch screen device may be the user device 404 with at least one surface upon which a user (e.g., a system administrator) may perform touch gestures (e.g., a laptop, a tablet computer, a personal digital assistant, a media player, a mobile phone, etc.).

As shown in FIG. 4A, the touch screen display 406 of the user device 404 displays a medicament administration graphical user interface (“GUI”) 408 provided by the handheld electronic computing device 106 and/or automated medicament delivery device 104, which, in some embodiments, can be accessible by the user device 404. For example, as described above with reference to FIG. 1, the handheld electronic computing device 106 can communicate with the automated medicament delivery device 104 via a network. As illustrated in FIG. 4A, in some embodiments, the medicament administration GUI 408 can include a time period content window 410, a desired therapy parameter content window 412, a correction content window 414, and an optional adjustments selectable element 416.

The time period content window 410 displays a selected time period that may be applicable to a selected at least one desired therapy parameter value within the desired therapy parameter content window 412 and includes one or more time period setting selectable elements 418. The time period setting selectable elements 418 may enable a user to choose starting and ending times of a time period applicable to the selected at least one desired therapy parameter value within the desired therapy parameter content window 412. The time period setting selectable elements 418 may include one or more of text input fields, radio buttons, checkboxes, dropdown menus, buttons, date-time pickers, or range sliders.

The desired therapy parameter content window 412 displays the at least one desired therapy parameter value and at least one desired therapy parameter value selectable element 420 for defining a target glucose value setting. The at least one desired therapy parameter value selectable elements 420 may include one or more of text input fields, radio buttons, checkboxes, dropdown menus, buttons, value pickers, or range sliders. As explained throughout this application, adjusting the desired target glucose value setting may cause a corresponding change to algorithm parameter values, such as the Q:R ratio or other parameters (e.g., as shown in Table 1), and such changes may impact the aggressiveness of drug delivery more than would otherwise be the case. This is so because the parameter values that may be changed based on a user's selected target glucose value are parameters that are conventionally not changed based on a target glucose value. Examples of parameter values that may be automatically adjusted based on the selected target blood glucose value are shown in Table 1 and discussed throughout this application.

The correction content window 414 provides a correction selectable element 422 that enables selection of a correction factor to adjust medicament dosage when blood glucose levels achieve levels higher than a target range. For instance, the correction selectable element 422 may include one or more of text input fields, radio buttons, checkboxes, dropdown menus, buttons, value pickers, or range sliders.

The optional adjustments selectable element 416 may allow a user to enable optional adjustments to input parameter values of the AID algorithm to achieve the at least one desired therapy parameter value. For instance, selection of the optional adjustments selectable element 416 may enable any of the optional adjustments described above in regard to FIG. 1 through FIG. 3. Furthermore, the optional adjustments selectable element 416 may allow the user to toggle between enabling the optional adjustments and disabling the optional adjustments. The optional adjustments selectable element 416 may include one or more of a text input field, radio button, a checkbox, a dropdown menu, a button, a value picker, or a range slider.

Referring to FIG. 4B, upon a selection of the optional adjustments selectable element 416, the medicament administration GUI 408 displays an information window 424. The information window 424 may display information regarding the consequences of enabling optional adjustments to input parameter values of the AID algorithm. Furthermore, responsive to selection of the optional adjustments selectable element 416, the controller 122 of the automated medicament delivery device 104 may perform any of the acts and operations described above in regard to act 306 through act 316 of the FIG. 3.

In the detailed description, reference is made to the accompanying drawings, which form a part hereof, and in which are shown, via illustration, specific examples of examples in which the present disclosure may be practiced. These examples are described in sufficient detail to enable a person of ordinary skill in the art to practice the present disclosure. However, other examples may be utilized, and structural, material, and process changes may be made without departing from the scope of the disclosure.

The illustrations presented herein are not meant to be actual views of any particular method, system, device, or structure, but are merely idealized representations that are employed to describe the examples of the present disclosure. The drawings presented herein are not necessarily drawn to scale. Similar structures or components in the various drawings may retain the same or similar numbering for the convenience of the reader; however, the similarity in numbering does not mean that the structures or components are necessarily identical in size, composition, configuration, or any other property.

The description may include examples to help enable one of ordinary skill in the art to practice the disclosed examples. The use of the terms “exemplary,” “by example,” and “for example,” means that the related description is explanatory, and though the scope of the disclosure is intended to encompass the examples and legal equivalents, the use of such terms is not intended to limit the scope of an embodiment or this disclosure to the specified components, steps, features, functions, or the like.

It will be readily understood that the components of the examples as generally described herein and illustrated in the drawing could be arranged and designed in a wide variety of different configurations. Thus, the description of various examples is not intended to limit the scope of the present disclosure, but is merely representative of various examples. While the various aspects of the examples may be presented in drawings, the drawings are not necessarily drawn to scale unless specifically indicated.

Furthermore, specific implementations shown and described are only examples and should not be construed as the only way to implement the present disclosure unless specified otherwise herein. Elements, circuits, and functions may be shown in block diagram form in order not to obscure the present disclosure in unnecessary detail. Conversely, specific implementations shown and described are exemplary only and should not be construed as the only way to implement the present disclosure unless specified otherwise herein. Additionally, block definitions and partitioning of logic between various blocks is exemplary of a specific implementation. It will be readily apparent to one of ordinary skill in the art that the present disclosure may be practiced by numerous other partitioning solutions. For the most part, details concerning timing considerations and the like have been omitted where such details are not necessary to obtain a complete understanding of the present disclosure and are within the abilities of persons of ordinary skill in the relevant art.

In the Brief Summary and in the Detailed Description, the claims, and in the accompanying drawings, reference is made to particular features (including method acts) of the present disclosure. It is to be understood that the disclosure includes all possible combinations of such particular features. For example, where a particular feature is disclosed in the context of a particular embodiment, or a particular claim, that feature can also be used, to the extent possible, in combination with and/or in the context of other particular aspects and examples described herein.

Those of ordinary skill in the art would understand that information and signals may be represented using any of a variety of different technologies and techniques. Some drawings may illustrate signals as a single signal for clarity of presentation and description. It will be understood by a person of ordinary skill in the art that the signal may represent a bus of signals, wherein the bus may have a variety of bit widths and the present disclosure may be implemented on any number of data signals including a single data signal.

The various illustrative methods, logical blocks, modules, and circuits described in connection with the examples of the system 100, and in particular, the automated medicament delivery device 104 and the handheld electronic computing device 106, disclosed herein may be implemented or performed with a general purpose processor, a special purpose processor, a digital signal processor (DSP), an Integrated Circuit (IC), an Application Specific Integrated Circuit (ASIC), a Field Programmable Gate Array (FPGA) or other programmable logic device, discrete gate or transistor logic, discrete hardware components, or any combination thereof designed to perform the functions described herein. A general-purpose processor (may also be referred to herein as a host processor or simply a host) may be a microprocessor, but in the alternative, the processor may be any conventional processor, controller, microcontroller, or state machine. A processor may also be implemented as a combination of computing devices, such as a combination of a DSP and a microprocessor, a plurality of microprocessors, one or more microprocessors in conjunction with a DSP core, or any other such configuration. A general-purpose computer including a processor is considered a special-purpose computer while the general-purpose computer is configured to execute computing instructions (e.g., software code) related to examples of the present disclosure.

The examples may be described in terms of a process that is depicted as a flowchart, a flow diagram, a structure diagram, or a block diagram. Although a flowchart may describe operational acts as a sequential process, many of these acts can be performed in another sequence, in parallel, or substantially concurrently. In addition, the order of the acts may be re-arranged. A process may correspond to a method, a thread, a function, a procedure, a subroutine, a subprogram, other structure, or combinations thereof. Furthermore, the methods disclosed herein may be implemented in hardware, software, or both. If implemented in software, the functions may be stored or transmitted as one or more instructions or code on computer-readable media. Computer-readable media includes both computer storage media and communication media including any medium that facilitates transfer of a computer program from one place to another.

All references cited herein are incorporated herein in their entireties. If there is a conflict between definitions herein and in an incorporated reference, the definition herein shall control.

The embodiments of the disclosure described above and illustrated in the accompanying drawings do not limit the scope of the disclosure, which is encompassed by the scope of the appended claims and their legal equivalents. Any equivalent embodiments are within the scope of this disclosure. Indeed, various modifications of the disclosure, in addition to those shown and described herein, such as alternate useful combinations of the elements described, will become apparent to those skilled in the art from the description. Such modifications and embodiments also fall within the scope of the appended claims and equivalents.

What is claimed is:

1. A system for administration of medicament to a user-body, the system comprising:
an analyte sensor; and
an automated medicament delivery device comprising:
at least one processor; and
at least one non-transitory computer-readable storage medium storing instructions thereon that, when executed by the at least one processor, cause the automated medicament delivery device to:
receive a user input selecting at least one desired therapy parameter value;
receive an additional user input enabling optional adjustments to input parameter values of an AID algorithm at least partially controlling delivery of medicament via the automated medicament delivery device;
responsive to receiving the additional user input, adjust one or more input parameters values of a set of initial input parameter values of the AID algorithm to create a set of adjusted input parameter values; and
generate a request to adjust administration of medicament to a user via the automated medicament delivery device utilizing at least a portion of the AID algorithm using the set of adjusted input parameter values.

2. The system for administration of claim 1, wherein the at least one desired therapy parameter value comprises a target blood glucose level.

3. The system for administration of claim 1, wherein the at least one desired therapy parameter value comprises at least one of a target pre-prandial glucose level, a TDI (Total Daily Insulin) value, a basal rate for a specific period of time, or a daily basal rate.

4. The system for administration of claim 1, wherein the set of initial input parameter values of the AID algorithm cause the AID algorithm to exhibit a first level of aggressiveness and wherein the set of adjusted input parameter values cause the AID algorithm to exhibit a second, different level of aggressiveness.

5. The system for administration of claim 4, wherein the second, different level of aggressiveness comprises an increased level of aggressiveness relative to the first level of aggressiveness.

6. The system for administration of claim 1, further comprising instructions that, when executed by the at least one processor, cause the automated medicament delivery device to, prior to receiving the additional user input enabling optional adjustments to input parameter values of the AID algorithm, deliver medicament to the user utilizing the AID algorithm using the set of initial input parameter values.

7. The system for administration of claim 1, further comprising instructions that, when executed by the at least one processor, cause the automated medicament delivery device to deliver medicament to the user according to the generated request.

8. The system for administration of claim 7, wherein causing the automated medicament delivery device to deliver medicament to the user according to the generated request comprises causing the automated medicament delivery device to deliver medicament to the user via least one of basal dosing or a bolus dose determined via the AID algorithm using the set of adjusted input parameter values.

9. The system for administration of claim 1, wherein, responsive to receiving the additional user input, adjust one or more input parameters values of a set of initial input parameters of the AID algorithm to create a set of adjusted input parameter values comprises automatically adjusting one or more input parameters values of the set of initial input parameters values of the AID algorithm based at least partially on the selected at least one desired therapy parameter value and without additional user input.

10. The system for administration of claim 1, wherein adjusting one or more input parameters values of the set of initial input parameters values of the AID algorithm comprises adjusting at least one of a Q:R Ratio, a TDI (Total Daily Insulin) value, an insulin-on-board (IOB) constraint duration of insulin action value, an insulin sensitivity factor (ISF) value, a carbohydrate ratio (CR), an amount of daily dose of long-acting insulin (LAI), amounts of anticipated bolus doses, carb count of meals, or a basal rate.

11. The system for administration of claim 10, wherein adjusting one or more input parameters values of the set of initial input parameters values of the AID algorithm comprises adjusting each of a Q:R Ratio, a TDI (Total Daily Insulin), and an insulin-on-board (IOB) constraint duration of insulin action value.

12. The system for administration of claim 1, wherein the initial input parameter values comprise one or more of a Q:R Ratio, a TDI (Total Daily Insulin) value, an insulin-on-board (IOB) constraint duration of insulin action value, an insulin sensitivity factor (ISF) value, a carbohydrate ratio (CR), an amount of daily dose of long-acting insulin (LAI), amounts of anticipated bolus doses, carb count of meals, a basal rate, or a target blood glucose level.

13. A method for managing medicament delivery to a user, the method comprising:
receiving, via a handheld electronic computing device, a user input selecting at least one desired therapy parameter value;
receive, via the handheld electronic computing device, an additional user input enabling optional adjustments to input parameter values of an AID algorithm at least partially controlling delivery of medicament via an automated medicament delivery device;
responsive to receiving the additional user input, adjust one or more input parameters values of a set of initial input parameter values of the AID algorithm to create a set of adjusted input parameter values; and
generate a request to adjust administration of medicament to a user via the automated medicament delivery device utilizing at least a portion of the AID algorithm using the set of adjusted input parameter values.

14. The method of claim 13, wherein the at least one desired therapy parameter value comprises a target blood glucose level.

15. The method of claim 13, wherein the at least one desired therapy parameter value comprises at least one of a target pre-prandial glucose level, a TDI (Total Daily Insulin) value, a basal rate for a specific period of time, or a daily basal rate.

16. The method of claim 13, wherein the set of initial input parameter values of the AID algorithm causes the AID algorithm to exhibit a first level of aggressiveness and wherein the set of adjusted input parameter values cause the AID algorithm to exhibit a second, different level of aggressiveness.

17. The method of claim 16, wherein the second, different level of aggressiveness comprises an increased level of aggressiveness relative to the first level of aggressiveness.

18. The method of claim 13, further comprising causing the automated medicament delivery device to, prior to receiving the additional user input enabling optional adjustments to input parameter values of the AID algorithm, to deliver medicament to the user utilizing the AID algorithm using the set of initial input parameter values.

19. The method of claim 13, wherein adjusting one or more input parameters values of the set of initial input parameters values of the AID algorithm comprises adjusting each of a Q:R Ratio, a TDI (Total Daily Insulin), and an insulin-on-board (IOB) constraint duration of insulin action value.

20. A system for administration of medicament to a user-body, the system comprising:
an analyte sensor; and
an automated medicament delivery device comprising:
at least one processor; and
at least one non-transitory computer-readable storage medium storing instructions thereon that, when executed by the at least one processor, cause the automated medicament delivery device to:
deliver medicament to a user utilizing an AID algorithm using a set of initial input parameter values;
receive a user input selecting at least one desired therapy parameter value;
receive an additional user input enabling optional adjustments to input parameter values of an AID algorithm at least partially controlling delivery of medicament via the automated medicament delivery device;
responsive to receiving the additional user input, adjust one or more input parameters values of the set of initial input parameter values of the AID algorithm to create a set of adjusted input parameter values;
generate a request to adjust administration of medicament to a user via the automated medicament delivery device utilizing at least a portion of the AID algorithm using the set of adjusted input parameter values; and
deliver medicament to the user according the generated request.
